(19) United States
(12) Patent Application Publication
(10) Pub. No.: US 2017/0028000 A1
(43) Pub. Date: Feb. 2, 2017

(54) USE OF LACTOBACILLUS RHAMNOSUS FOR PROMOTING RECOVERY OF THE INTESTINAL MICROBIOTA DIVERSITY AFTER DYSBIOSIS

(71) Applicants: COMPAGNIE GERVAIS DANONE, Paris (FR); INSTITUT NATIONAL DE LA RECHERCHE AGRONOMIQUE, Paris (FR)

(72) Inventors: Gianfranco Grompone, Paris (FR); Muriel Derrien, Bures Sur Yvette (FR); Johan Van Hylckama Vlieg, Chavenay (FR); Pascale Serror, Jouy-en-josas (FR); Lionel Rigottier-Gois, Palaiseau (FR); Laureen Crouzet, Blanzat (FR); Claire Cherbuy, Les Ulis (FR)

(73) Assignees: COMPAGNIE GERVAIS DANONE, Paris (FR); INSTITUT NATIONAL DE LA RECHERCHE AGRONOMIQUE, Paris (FR)

(21) Appl. No.: 15/303,285

(22) PCT Filed: Apr. 15, 2015
(86) PCT No.: PCT/IB2015/052753

§ 371 Date: Oct. 11, 2016

(30) Foreign Application Priority Data
Apr. 15, 2014 (IB) PCT/IB2014/060742

(51) Int. Cl. A61K 35/747

(52) U.S. Cl. CPC A61K 35/747; A61K 2035/11

(57) ABSTRACT

The present invention provides the use of Lactobacillus rhamnosus, for maintaining or increasing the intestinal microbiota diversity in a subject.

### FIELD OF THE INVENTION

The present invention relates to the field of probiotics. In particular, the invention pertains to the use of Lactobacillus rhamnosus, to maintain or increase the intestinal microbiota diversity in a subject.

### BACKGROUND

According to a definition approved by a joint Food and Agriculture Organization of the United Nations/World Health Organization (FAO/WHO) expert Consultation on Health and Nutritional properties of powder milk with live lactic acid bacteria in 2001, probiotics are “live microorganisms which when administered in adequate amounts confer a health benefit on the host”. Probiotic bacteria have been described among species belonging to the genera Lactobacillus, Bifidobacterium, Streptococcus and Lactococcus, which are commonly used in the dairy industry. Probiotics are thought to intervene at the level of the gut microbiota by impeding the development of pathogenic microorganisms and/or by acting more directly on the immune system.

Opportunistic bacterial infections responsible for healthcare associated infections (HAIs) contribute significantly to patient mortality and morbidity, as well as healthcare costs both in developed and developing countries (WHO, 2008). The gastrointestinal tract (GIT) is a reservoir for opportunistic pathogens, which benefit from the disruption of the intestinal microbiota balance, or dysbiosis, to invade and infect susceptible patients. In particular, antibiotic treatments have deleterious effects on the diversity of the intestinal microbiota and they promote expansion of bacterial human opportunistic pathogens including Enterococcus faecalis, Enterococcus faecium or Clostridium difficile.

Having acquired antibiotic resistance and other pathogenic traits, multi-drug resistant colonizing and/or invasive E. faecalis isolates, which cause serious nosocomial infections, are grouped in seven hospital-adapted complexes designated as High-Risk Enterococcal Clonal Complexes (HiRECCs). Proliferation and persistence of HiRECCs within the GIT are a major risk of developing a vancomycin-resistant enterococcal (VRE) infection, highlighting a need for a better understanding of the biological and biochemical factors involved in colonization of the GIT by E. faecalis. Isolates belonging to HiRECC-2 are among the most common causes of E. faecalis infections in the United States and in several European countries.

It is clear from the above that there is a need for alternatives or complements to antibiotics for the treatment or for the prevention of E. faecalis infection.

The “gut microbiota” designates the population of microorganisms living in the intestine of any organism belonging to the animal kingdom (human, animal, insect, etc.). While each individual has a unique microbiota composition (60 to 80 bacterial species are shared by more than 50% of a sampled population of a total of 400-500 different bacterial species/individual), it always fulfils similar main physiological functions and has a direct impact on the individual's health:
it contributes to the digestion of certain foods that the stomach and small intestine are not able to digest (mainly non-digestible fibers);
it contributes to the production of some vitamins (B and K);
it protects against aggressions from other microorganisms, maintaining the integrity of the intestinal mucosa;
it plays an important role in the development of a proper immune system;
a healthy, diverse and balanced gut microbiota is key to ensuring proper intestinal functioning.

Taking into account the major role that gut microbiota plays in the normal functioning of the body and the different functions it accomplishes, it is sometimes considered to be an “organ”. However, it is an “acquired” organ, as babies are born sterile; that is, intestine colonization starts at birth and evolves afterwards.

The magnitude of disturbance of the gut microbiota following a perturbation such as a dietary change, an antibiotic treatment and an invasion by an exogenous microbe, and the speed and extent of the recovery to the pre-perturbation state, was defined as “the resilience of the microbiota”. Resilience of the microbiota varies across individuals and between different perturbations within an individual.

From the above, it appears that there is also an important need for treatments for increasing the resilience of the microbiota.

Growing evidence shows that probiotics or fecal microbiota transplantation prevent or treat a number of diseases, including intestinal infections. Such approaches were also associated with higher clearance of intestinal VRE in mice.

Surprisingly the inventors have found that the bacterial species Lactobacillus rhamnosus is capable of promoting recovery of intestinal microbiota diversity.

Accordingly, a subject of the present invention is the use of Lactobacillus rhamnosus, for increasing the resilience of the gut microbiota. In particular, the present invention pertains to the use of Lactobacillus rhamnosus, to maintain or increase the intestinal microbiota diversity of a subject. In a particular embodiment, the use of Lactobacillus rhamnosus allows to maintain or increase the intestinal microbiota diversity of a subject having an intestinal dysbiosis caused by antibiotics.

Further aspects of the present invention provide the use of Lactobacillus rhamnosus in the prevention, reduction or treatment of intestinal dysbiosis; and/or prevention of a disease caused by a pathogen present in the gastrointestinal tract; and/or increase in the level of short-chain fatty acid in a subject.

The invention also provides compositions comprising Lactobacillus rhamnosus for use according to the present invention.

### DETAILED DESCRIPTION

In the present text, the phrases “maintain the microbiota diversity” will be used to express that species diversity (species richness and/or species evenness) of the microbiota of an individual will not be significantly modified or affected, especially in case of dysbiosis. In particular, maintaining the microbiota diversity could help the subject to recover faster in case of risk of dysbiosis or could avoid the dysbiosis to worse. The phrases “increase of microbiota diversity”, “promote recovery of microbiota diversity”, “treatment/decrease/reduction/of dysbiosis” etc. will be used to express an increase in species diversity (species richness and/or species evenness) of the microbiota of an individual. Methods for the calculation of species diversity, species richness and species evenness are known in the art and include but are not limited to Simpson's Index, Simpson's Index of Diversity and Simpson's Reciprocal Index, Chao Index and Shannon Index.

In addition, “accelerate the increase of the intestinal microbiota diversity”, “promote recovery of the intestinal microbiota diversity”, “favour the return to a baseline/normal/healthy intestinal microbiota diversity”, “accelerate the decrease/reduction/disappearance of the dysbiosis” etc. will be used to express that the diversity (richness and/or evenness) of the microbiota of individuals having an intestinal dysbiosis after a treatment by antibiotics increases statistically more rapidly in subjects who take the probiotic strain than in control subjects who do not, so that the structure of the microbiota three weeks after the antibiotic treatment is statistically closer to the structure before said treatment in subjects who take the probiotic strain than in control subjects who do not.

As used herein the term “dysbiosis” shall be taken to mean a change in microbiota commensal species diversity as compared to a healthy or general population and shall include decrease of beneficial microorganisms and/or increase of pathobionts (pathogenic or potentially pathogenic microorganisms) and/or decrease of overall microbiota species diversity. Many factors can harm the beneficial members of the intestinal microbiota leading to dysbiosis, including antibiotic use, psychological and physical stress, radiation, and dietary changes. Antibiotic use is the most common and significant cause of major alterations in normal microbiota. Thus, as used herein, the term “antibiotic-induced dysbiosis”refers to dysbiosis caused by antibiotic comprising the promotion of overgrowth of bacterial opportunistic pathogens including Enterococcus faecalis, Enterococcus faecium or Clostridium difficile.

As used herein the term “dairy composition” shall be taken to mean a milk-based composition suitable for animal consumption, in particular human consumption.

As used herein the term “milk” shall be taken to include vegetal or animal milk, such as but not limited to soya, almond, spelt, oat, hemp, coconut, rice, goat, ewe, or cow milk.

As used herein the term “x % (w/w)” is considered equivalent to “x g per 100 g”.

As used herein reference to a bacterial strain or species shall be taken to include bacteria derived therefrom wherein said bacteria retain the capacity to decrease intestinal dysbiosis of a subject, preferably a subject having an antibiotic-induced dysbiosis. To assess this capacity, the same model as described in the Examples below can be used. Strains derived from a parent strain which can be used according to the present invention include mutant strains and genetically transformed strains. These mutants or genetically transformed strains can be strains wherein one or more endogenous gene(s) of the parent strain has (have) been mutated, for instance to modify some of their metabolic properties (e.g., their ability to ferment sugars, their resistance to acidity, their survival to transport in the gastrointestinal tract, their post-acidification properties or their metabolite production). They can also be strains resulting from the genetic transformation of the parent strain to add one or more gene(s) of interest, for instance in order to give to said genetically transformed strains additional physiological features, or to allow them to express proteins of therapeutic or vaccinal interest that one wishes to administer through said strains. These mutants or genetically transformed strains can be obtained from the parent strain by means of conventional techniques for random or site-directed mutagenesis and genetic transformation of bacteria, or by means of the technique known as “genome shuffling”. In the present text, strains, mutants and variants derived from a parent species or strain, and retaining the ability to maintain or increase intestinal microbiota diversity of a subject having an antibiotics-induced dysbiosis will be considered as being encompassed by reference to said parent species or strain, e.g. the phrases “Lactobacillus rhamnosus” and “strain CNCM I-3690” shall be taken to include strains, mutants and variants derived therefrom.

As used herein the term “food supplement” shall be taken to mean a product made from compounds usually used in foodstuffs, but which is in the form of tablets, powder, capsules, potion or any other form usually not associated with aliments, and which has beneficial effects for one's health.

As used herein the term “functional food” shall be taken to mean an aliment which has beneficial effects for one's health in addition to providing nutrients. In particular, food supplements and functional food can have a physiological effect—for the prophylaxis, amelioration or treatment of a disease, for example a chronic disease.

As used herein the term “fermented dairy” or “fermented milk” refers to a composition derived from a dairy or milk composition respectively by the acidifying action of at least one lactic acid bacterium, which may be comprised in a ferment, a culture or a starter.

As used herein the term “spoonable” shall be taken to mean a solid or semi-solid that may be consumed by means of a spoon or other utensil.

Uses of L.rhamnosus

The present invention provides the use of L. rhamnosus, preferably strain CNCM I-3690, for use to maintain or increase the intestinal microbiota diversity in a subject, preferably a subject having intestinal dysbiosis.

Accordingly, in one embodiment the present invention provides the use of L. rhamnosus, preferably strain CNCM I-3690, for the prevention or decrease of intestinal dysbiosis in a subject. Strain CNCM I-3690 was deposited, according to the Budapest Treaty, at CNCM (Collection Nationale de Cultures de Microorganismes, 25 rue du Docteur Roux, Paris) on Nov. 9, 2006. This strain is disclosed in International Application WO 2009/122042.

In a preferred embodiment the intestinal dysbiosis is caused by or subsequent to antibiotic treatment of the subject.

In a further embodiment the present invention provides the use of Lactobacillus rhamnosus, preferably strain CNCM I-3690, for preventing a gastrointestinal bacterial infection and/or the development of a disease caused by a pathogen present in the gastrointestinal tract, preferably a commensal and/or opportunistic pathobiont. Such a disease can be localized in the GIT, or extend to the abdominal cavity, blood, etc. in case the opportunist pathogen crosses the intestinal barrier (said crossing being favoured by a significant and/or long dysbiosis). Accordingly, the present invention further provides a method for preventing a gastrointestinal bacterial infection and/or the development of a disease caused by a pathogen present in the gastrointestinal tract in a subject, comprising administering an effective amount of a composition comprising L. rhamnosus, preferably strain CNCM I-3690, to the subject. Preferably the subject has intestinal dysbiosis. In a preferred embodiment, the intestinal dysbiosis is caused by or subsequent to antibiotic treatment of the subject. In a further preferred embodiment, the pathogen is Enterococcus faecalis.

Increase in Short-Chain Fatty Acid

The inventors have also shown that, in parallel to the maintaining or increase of microbiota diversity, administration of Lactobacillus rhamnosus, preferably strain CNCM I-3690, leads to an increase in the level of short-chain fatty acids in a subject. As previously described in the art, a broad diversity in microbiota and a high level of SCFA, especially of butyrate, in microbiota are favourable to health, alone or in association. Thus, the fact that Lactobacillus rhamnosus allows to increase the microbiota diversity and to increase SCFA in case of dysbiosis, indicate that Lactobacillus rhamnosus could particularly be health beneficial.

Accordingly in a further embodiment the present invention provides the use of Lactobacillus rhamnosus, preferably strain CNCM I-3690, for increase in short-chain fatty acid in a subject. In one embodiment said short-chain fatty acid is intestinal, typically colon, distal colon, caecal or fecal. In a preferred embodiment said short-chain fatty acid is butyrate. In an alternative embodiment the present invention provides the use of Lactobacillus rhamnosus, preferably strain CNCM I-3690, for increase of the butyrate/acetate ratio. Preferably the subject has intestinal dysbiosis. In a further preferred embodiment the intestinal dysbiosis is caused by or subsequent to antibiotic treatment of the subject.

Compositions

A further aspect of the present invention provides compositions comprising L. rhamnosus, preferably strain CNCM I-3690, for uses according to the present invention. Thus, the present invention provides compositions comprising L. rhamnosus, preferably strain CNCM I-3690, for use to maintain or increase the intestinal microbiota diversity; and/or the prevention or treatment of intestinal dysbiosis; and/or prevention of a disease caused by a pathogen present in the gastrointestinal tract, preferably a commensal and/or opportunistic pathobiont; and/or increase in the level of short-chain fatty acid in a subject. Preferably, said subject has intestinal dysbiosis, it is further preferred that said dysbiosis is caused by or subsequent to antibiotic treatment of the subject.

Accordingly in a preferred embodiment of the present invention, the strain L. rhamnosus, preferably strain CNCM I-3690, is provided as an orally administrable composition. In such a composition, said strain can be used in the form of whole bacteria which may be living or dead. Alternatively, said strain can be used in the form of a bacterial lysate. Preferably, the bacterial cells are present as living and viable cells.

According to the present invention, the composition can be in any form suitable for oral administration. This includes for instance solids, semi-solids, liquids, and powders. Semi-solid compositions, such as yogurts, and liquid compositions, such as drinks, are preferred.

The composition preferably comprises at least 1.10⁶ colony forming units (cfu), at least 1.10⁷ colony forming units (du) or preferably at least 1.10⁸ cfu per gram weight, of L. rhamnosus, preferably the strain CNCM I-3690. Preferably also the composition according to the invention comprises up to about 10¹¹, more preferably at least 10¹⁰ and most preferably at least 10⁹ colony forming unit (CFU) of L. rhamnosus, preferably the strain CNCM I-3690, according to the invention per gram (g) of composition according to the invention.

The composition can further comprise other strains of Lactobacillus and/or other strains of bacteria than the strains mentioned above, in particular probiotic strain(s), such as Streptococcus thermophilus, Bifidobacterium and Lactococcus strain(s).

The composition can be a pharmaceutical composition or a nutritional composition. According to a preferred embodiment, the composition is a nutritional composition such as a food product (including a functional food) or a food supplement.

Nutritional compositions which can be used according to the invention include dairy compositions, preferably fermented dairy compositions. The fermented compositions can be in the form of a liquid or in the form of a dry powder obtained by drying the fermented liquid. Examples of dairy compositions include fermented milk and/or fermented whey in set, stirred or drinkable form, cheese and yoghurt. The fermented product can also be a fermented vegetable, such as fermented soy, cereals and/or fruits in set, stirred or drinkable forms. Nutritional compositions which can be used according to the invention also include baby foods, infant milk formulas and infant follow-on formulas. In a preferred embodiment, the fermented product is a fresh product. A fresh product, which has not undergone severe heat treatment steps, has the advantage that the bacterial strains present are in the living form.

It is particularly preferred that the composition according to the invention is a dairy composition, in particular a fermented dairy composition.

Preferably, the dairy composition according to the invention comprises or derives (in particular by fermentation) from a composition containing from 30 to 100% (w/w) milk, more preferably from 50 to 100% (w/w) milk and even more preferably from 70 to 100% (w/w) milk. Preferably also, the dairy composition according to the invention comprises or derives (in particular by fermentation) from a composition essentially consisting of milk or consisting only of milk, preferably to cow milk.

Preferably, the dairy composition according to the invention comprises or derives (in particular by fermentation) from a composition comprising one or both of skimmed or non-skimmed milk. Preferably said milk or milks may be in liquid, powdered and/or concentrated form. In one embodiment said milk or milks may be enriched or fortified with further milk components or other nutrients such as but not limited to vitamins, minerals, trace elements or other micronutrients.

The fermented dairy composition is derived from a dairy composition according to the invention by the acidifying action of at least one lactic acid bacterium, which may be comprised in a ferment, a culture or a starter. More preferably said fermented dairy composition according to the invention is obtained by the acidifying action of at least one, two, three, four, five, six, seven or more lactic acid bacteria strains. Accordingly the “fermented dairy composition” comprises at least one, two, three, four, five, six, seven or more lactic acid bacteria strains.

Methods for the preparation of fermented milk products, such as yogurts or equivalents thereof, are well-known in the art. Typically a fermented milk product is prepared by culture of heat-treated (e.g. pasteurized) skimmed and/or non-skimmed milks with suitable microorganisms to provide a reduction in pH. The selection of suitable microorganisms (e.g. thermophilic lactic acid bacteria) is within the scope of the skilled person.

The dairy composition, in particular the fermented dairy composition, according to the invention, may optionally further comprise secondary ingredients such as fruits, vegetables, nutritive and non-nutritive sweeteners, cereals, flavours, starch, thickeners, preservatives or stabilizers. Preferably the dairy composition, in particular the fermented dairy composition, according to the invention shall comprise up to about 30% (w/w) of said secondary ingredients, e.g. up to about 10%, 15%, 20%, 25% (w/w).

Preferably, the dairy composition according to the invention is a fermented dairy composition, more preferably a fermented milk composition that comprises, comprises essentially of or consists of milk that has been subjected to heat treatment at least equivalent to pasteurization, preferably said heat treatment is carried out prior to the preparation of the dairy composition or fermented dairy composition.

Preferably, the dairy composition according to the invention is a fermented dairy composition, more preferably a fermented milk composition that comprises above about 0.3 g per 100 g by weight free lactic acid, more preferably the invention provides a fermented milk composition comprising above about 0.7 g or 0.6 g per 100 g by weight free lactic acid.

Preferably the dairy composition according to the invention is a fermented dairy composition, more preferably a fermented milk composition that comprises a protein content at least equivalent to that of the milk or milks from which it is derived.

Preferably, the dairy composition according to the invention is a fermented dairy composition, more preferably a fermented milk composition that has a pH equal to or lower than 5, more preferably between about 3.5 and about 4.5.

Preferably, the dairy composition according to the invention is a fermented dairy composition, more preferably a fermented milk composition that has a viscosity lower than 200 mPa·s, more preferably lower than 100 mPa·s and most preferably lower that 60 mPa·s, at 10° C., at a shear rate of 64 s-1. In one embodiment the dairy composition according to the invention is a drinkable fermented dairy composition, more preferably a drink fermented milk drink such as but not limited to a yogurt drink, kefir etc.. In an alternative embodiment the dairy composition according to the invention is a fermented dairy composition, more preferably a fermented milk composition that is spoonable.

Preferably also, the dairy composition, in particular the fermented dairy composition, according to the invention, or the product according to the invention, may be stored at a temperature of from 1° C. to 10° C.

A single serving portion of the dairy composition, in particular the fermented dairy composition according to the invention, more preferably a fermented milk composition or the product according to the invention is preferably about 50 g, 60 g, 70 g, 75 g, 80g, 85 g,90 g, 95 g, 100 g, 105 g, 110 g, 115 g, 120g, 125 g, 130 g, 135 g, 140g, 145 g, 150 g, 200 g, 300 g or 320 g or alternatively about 1 oz, 2 oz, 3 oz, 4 oz, 5 oz, 6 oz or 12 oz by weight.

Preferably, the dairy composition, in particular the fermented dairy composition according to the invention, more preferably a fermented milk composition according to the invention comprises at least 10⁶, more preferably at least 10⁷ and most preferably at least 10⁸ colony forming unit (CFU) of L. rhamnosus, preferably the strain CNCM I-3690, according to the invention per gram (g) of composition according to the invention.

Therapeutic Uses

A subject of the present invention is also the use of L. rhamnosus, preferably the strain CNCM I-3690, or a composition as defined above, for the manufacture of a medicament for the maintaining or increase of intestinal microbiota diversity; and/or prevention or treatment of intestinal dysbiosis; and/or prevention of a disease caused by a pathogen present in the gastrointestinal tract, preferably a commensal and/or opportunistic pathobiont; and/or increase in the level of short-chain fatty acid in a subject. Preferably, said subject has intestinal dysbiosis, it is further preferred that said dysbiosis is caused by or subsequent to antibiotic treatment of the subject.

A subject of the present invention is also the use of a L. rhamnosus strain as defined above, preferably the strain CNCM I-3690, or a composition as defined above for use to maintain or increase the intestinal microbiota diversity; and/or in the prevention or treatment of intestinal dysbiosis; and/or prevention of a disease caused by a pathogen present in the gastrointestinal tract, preferably a commensal and/or opportunistic pathobiont; and/or increase in the level of short-chain fatty acid in a subject. Preferably said subject has intestinal dysbiosis, it is further preferred that said dysbiosis is caused by or subsequent to antibiotic treatment of the subject.

A subject of the present invention is also a method for the maintaining or increase of intestinal microbiota diversity; and/or prevention or treatment of intestinal dysbiosis; and/or prevention of a disease caused by a pathogen present in the gastrointestinal tract, preferably a commensal and/or opportunistic pathobiont; and/or increase in the level of short-chain fatty acid in a subject in need thereof, said method comprising administering to said subject a therapeutically effective amount of a L. rhamnosus strain as defined above, preferably the strain CNCM I-3690, or a composition as defined above. Preferably said subject has intestinal dysbiosis, it is further preferred that said dysbiosis is caused by or subsequent to antibiotic treatment of the subject.

Determination of a therapeutically effective amount is well known by the person skilled in the art, especially in view of the detailed disclosure provided herein.

A subject of the present invention is also a method for the manufacture of a medicament for the maintaining or increase of intestinal microbiota diversity; and/or prevention or treatment of intestinal dysbiosis; and/or prevention of a disease caused by a pathogen present in the gastrointestinal tract, preferably a commensal and/or opportunistic pathobiont; and/or increase in the level of short-chain fatty acid, said method comprising incorporating a L. rhamnosus strain as defined above, preferably the strain CNCM I-3690 into at least one pharmaceutically acceptable diluent, carrier or excipient.

Dosage

In one embodiment, the present invention provides the consumption or administration of a dose of between about 10⁸ and about 10¹¹ colony forming unit (CFU) of L. rhamnosus, preferably between about 10⁸ and about 10⁹, more preferably between about 10⁹ and about 10¹⁰ colony forming unit (CFU) and in an alternative embodiment between about 10¹⁰ and about 10¹¹ colony forming unit (CFU) of L. rhamnosus, more preferably strain CNCM I-3690 for the uses and methods as disclosed herein. In a further embodiment at least 1, 2, 3, or 4 doses are provided within a 24 hour time period. It is further preferred that the daily dosage regimen is maintained for at least about 1, 2, 3, 4, 5, 6 or 7 days, or in alternative embodiment for at least about 1, 2, 3, 4, 5, 6 or 7 weeks.

Accordingly, in one embodiment the present invention provides the daily consumption or administration of at least 1, 2, 3, or 4 servings of the dairy composition, in particular the fermented dairy composition according to the invention, more preferably a fermented milk composition according to the invention. Each serving may be consumed or administered individually, or a plurality of servings may be consumed or administered in a single instance. Each of said servings may be consumed at mealtimes or between mealtimes (e.g. as a snack, subsequent to sporting activities etc . . . ).

A single serving portion of the dairy composition, in particular the fermented dairy composition according to the invention, more preferably a fermented milk composition, according to the invention is preferably about 50 g, 60 g, 70 g, 75 g, 80 g, 85 g, 90 g, 95 g, 100 g, 105 g, 110 g, 115g, 120 g, 125 g, 130 g, 135 g, 140 g, 145 g, 150 g, 200 g, 300 g or 320 g or about 1 oz, 2 oz, 3 oz, 4 oz, 5 oz, 6 oz or 12 oz by weight.

Preferably, the composition according to the invention comprises at least 10⁶, more preferably at least 10⁷ and most preferably at least 10⁸ colony forming unit (CFU) of >L. rhamnosus, more preferably strain CNCM I-3690, according to the invention per gram (g) of composition according to the invention. Preferably also the composition according to the invention comprises the at least 10¹¹, more preferably at least 10¹⁰ and most preferably at least 10⁹ colony forming unit (CFU) of L. rhamnosus, more preferably strain CNCM I-3690, bacteria per gram (g) of composition according to the invention.

For example, in one embodiment the present invention provides the daily consumption of at least 2 or at least 3 servings of a 100g or 125 g portion of a fermented milk product comprising between about at least 10⁷ and at least 10⁸ colony forming units (CFU) L. rhamnosus I-3690 per g product. In a further embodiment said daily level of consumption is maintained over a period of at least 1, 2, 3, 4 or more weeks.

The present invention will be understood more clearly from the further description which follows, which refers to examples illustrating the capacity of the L. rhamnosus, for example strain CNCM I-3690 of decreasing dysbiosis in vivo and increasing short-chain fatty acid, as well as to the appended figures.

### BRIEF DESCRIPTION OF THE DRAWINGS

FIG. 1: Scheme representing the sequence of the experiments.

FIG. 2: Richness of microbiota measured by Chao index. C: Control, Lr: L. rhamnosus.

FIG. 3: Relative abundance of Enterococcus and Enterobacteriaceae at baseline, after clindamycin treatment and restoration.

FIG. 4: Principal Coordinates analysis of weighted Unifrac distances of samples collected at D0, D7, D10, D11 and D21 from control and L. rhamnosus group (n=3 per group and time point).

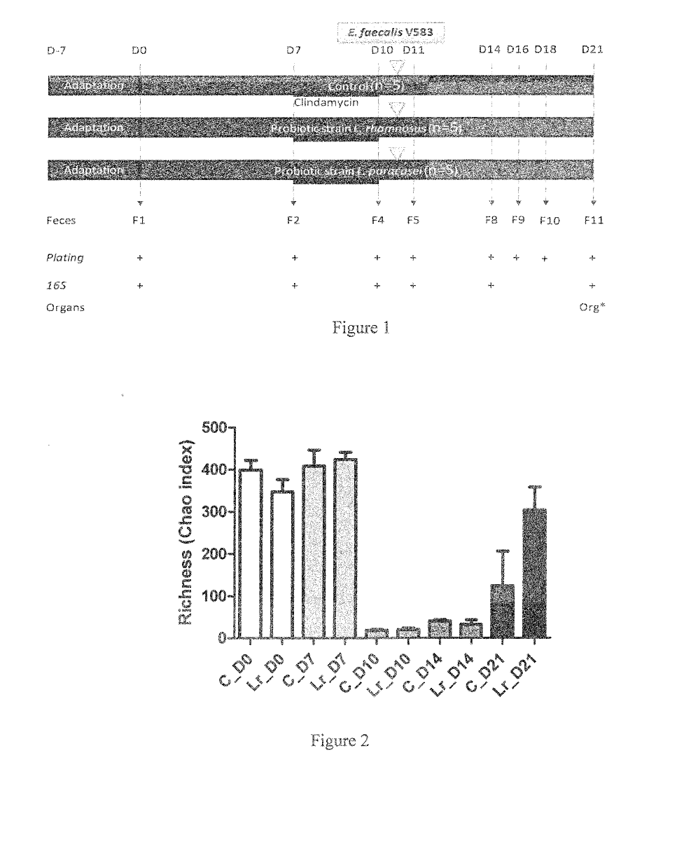

### EXAMPLES

The inventors developed an intestinal colonization mouse model based on a microbiota dysbiosis induced by clindamycin to mimic enterococci overgrowth and VRE establishment. Mice received subcutaneous clindamycin for 3 days before or gastric inoculation with Enterococcus faecalis VRE strain (V583). The native microbiota in mice is nearly or totally devoid of Enterococcus faecalis; moreover, the commensal-to-pathogen switch does not happen in mice. Using this model, probiotic strains were daily orally administered to mice starting one week before antibiotic treatment until two weeks after arrest of antibiotic treatment and inoculation of VRE. Kinetics of establishment and clearance of VRE as well of indigenous enterococci population levels were monitored by selective plating. In parallel, fecal samples were collected for 16S rRNA gene survey analysis of the whole microbiota. The dysbiosis induced in this model mimics the antibiotic-induced dysbiosis observed in humans. This model hence constitutes a good model to study the mechanisms of intestinal colonization barrier against enterococci overgrowth. The strain V583 belongs to CC2 and was the first vancomycin resistant isolate reported in the United States (Sahm et al., 1989). This strain was used in the experiments reported below as a model strain of CC2 isolates and more generally, of pathogenic E. faecalis.

Methods

Bacterial Growth

E. faecalis V583 strain was grown in M17 supplemented with 0.5% glucose (GM17) and collected by centrifugation 1 h after reaching stationary phase. Bacterial cells were washed twice with 0.9% saline solution and stored as a dry frozen pellet at −80° C. This strain belongs to CC2 and was the first vancomycin resistant isolate reported in the United States (Sahm et al., 1989).

Probiotic strains were grown in MRS media, and collected as describe above.

At least two days before inoculation, the frozen bacteria were suspended in a saline solution and serial dilutions were plated on GM17 or MRS agar plates to determine the bacterial count of the pellet.

Mouse E. faecalis Model Colonization

Mouse experiments were performed using specific pathogen-free male CF-1 mice (Harlan, USA), 6-8-weeks. A total of 5 mice were housed in each cage and were fed with autoclaved food and water ad libitum.

They received a daily dose of 10⁹ CFU of probiotic strain in 0.1 ml of 0.9% saline solution by or gastric inoculation using a steel feeding tube (Ecimed). Lactobacillus rhamnosus I-3690 was administered to the Lr group and Lactobacillus rhamnosus I-3689 for the Lp group. Animals from the control group received 0.1 ml of 0.9% saline solution by the same way. After one week of probiotic treatment, a dose of 1.4 mg/day of clindamycin was administered subcutaneously daily for three days. One day later, 10¹⁰ colony-forming units (CFU) of E. faecalis (vancomycin-resistant enterococci, noted “VRE”) strain V583 in 0.1 ml of 0.9% saline solution were administered by or gastric inoculation using a steel feeding tube (Ecimed).

Stool samples were collected as depicted in the experimental design below. Feces (from 50 to 100 mg/mice) or ceca were kept at 4° C. and were treated within 3 hours after sampling and processed at room temperature. From this stage, all the work done was performed in sterile conditions under PSMII. Samples were weighted and suspended at a dilution of 10⁴. An adjusted volume of peptone water was added according to the weight (eg., 900 μl for 100 mg, 450 μl for 50 mg). A volume of 100 μl of the suspension (dilution −1) was used to perform decimal dilutions in peptone water until 10⁻⁸. Total enterococci population were monitored by plating diluted stool samples onto BEA, and total lactobacilli on MRS media, and then incubated 48 h at 37° C. under anaerobic condition (Gaz pack). For the study with E. faecalis V583 administration, the population level of V583 was followed by plating onto BEA supplemented with vancomycin at 6 μg/mL. Fecal samples were also collected for 16S rRNA gene survey analysis of the whole microbiota. At the end of the experiment, the animals were sacrificed. Cecal contents were collected to assess fermentation patterns by measuring concentrations of short chain fatty acid. Colons were recovered and immediately used for RNA extraction (frozed in liquid nitrogen) or histology (paraformaldehyde solution 4%).

Microbiota Analysis

Faecal samples were collected at DO (baseline), D7 (1 week probiotic treatment), D10 (3 days antibiotics intake), D11 (1 day post E. faecalis V583 inoculation) and D21 (sacrifice). DNA was extracted using Godon et al procedure (Godon, 1997). For pyrosequencing, V3-V5 region of the 16S rRNA gene was amplified using key-tagged eubacterial primers (Lifesequencing S.L., Valencia, Spain) based on design of Sim et al 2012. PCR reactions were performed with 20 ng of metagenomic DNA, 200 μM of each of the four deoxynucleoside triphosphates, 400 nM of each primer, 2.5 U of FastStart HiFi Polymerase, and the appropriate buffer with MgCl₂ supplied by the manufacturer (Roche, Mannheim, Germany), 4% of 20 g/mL BSA (Sigma, Dorset, United Kingdom), and 0.5 M Betaine (Sigma). Thermal cycling consisted of initial denaturation at 94° C. for 2 minutes followed by 35 cycles of denaturation at 94° C. for 20 seconds, annealing at 50° C. for 30 seconds, and extension at 72° C. for 5 minutes. Amplicons were combined in a single tube in equimolar concentrations. The pooled amplicon mixture was purified twice (AMPure XP kit, Agencourt, Takeley, United Kingdom) and the cleaned pool requantified using the PicoGreen assay (Quant-iT, PicoGreen DNA assay, Invitrogen). Subsequently, an amplicon submitted to the pyrosequencing services offered by Life Sequencing S.L. (Valencia, Spain) where EmPCR was performed and subsequently, unidirectional pyrosequencing was carried out on a 454 Life Sciences GS FLX+ instrument (Roche) following the Roche Amplicon Lib-L protocol. Bioinformatic analyses were performed using QIIME v.1.6 (Caporaso, 2010). Data were assigned to 50 samples after filtering according to the following quality criteria: size between 500 and 1000 nt, quality above 25 over a 50 base pairs window, no mismatch authorized in primers and barcode sequences, and absence of polymers larger than 6 nt. Remaining reads were clustered into Operational Taxonomic Units (OTUs) defined at 97% identity using cd-hit (Li, 2006) and representative sequences for each OTU were aligned and taxonomically assigned using Greengenes v_13_08 database. For alpha and beta diversity, samples were rarefied to 3000 sequences per sample. Alpha-diversity (that measures diversity within samples) was assessed using rarefaction curves for richness (Chao 1), and evenness (Shannon index) and numbers of observed OTUs. Beta diversity (that measures diversity between samples) was performed on both weighted and unweighted Unifrac distances using 3500 reads.

Cecal Fermentation End Products Measurement

The concentrations of the short chain fatty acids (SCFAs), including acetate, propionate and butyrate concentrations were determined using 500 mg caecal content supernatants after water extraction of acidified samples using gas liquid chromatography (Nelson 1020, Perkin-Elmer, St Quentin en Yvelines, France) as described previously (Lan et al, 2008). Lactate was determined using D-L lactic-acid kit (BioSenTeck).

Statistical Analysis

Differences in bacterial counts, microbial diversity (richness and evenness) and short chain fatty acid data were analyzed by the Mann-Whitney test (GraphPad). Differences were considered significant when P<0.05.

Results: Strain L. rhamnosus I-3690 Promotes Recovery of Microbiota Diversity and Increases Caecal Butyrate/Acetate Ratio After Dysbiosis in the Presence of E. faecalis V583

In the E. faecalis colonization model, transient increase of indigenous enterococci is concomitant with clindamycin treatment. Enterococci population reaches the highest level one day after the arrest of antibiotic treatment and then decreases progressively to the initial level 4 to 5 days later. After inoculation, the Enterococcus faecalis VU strain parallels indigenous enterococci and persists at detectable level at least up to 11 days post-gavage (Rigottier-Gois et al. submitted). In this project, microbiota analysis using 454 pyro sequencing of bacterial 16S rRNA gene revealed that overgrowth of indigenous enterococci correlated with decreased microbiota diversity resulting from antibiotic treatment. The administration of the probiotic strains had no effect on enterococci overgrowth (FIG. 2).

To profile the effects of clindamycin treatment +VRE inoculation, and L. rhamnosus CNCM I-3690 consumption on microbiota structure, 454 pyrosequencing of bacterial 16S rRNA gene V3-V5 variable regions was performed on fecal samples collected from mice at DO (baseline), D7 (1 week probiotic consumption), D10 (3 days clindamysin treatment), D11 (E. faecalis VRE inoculation) and D21 (“restoration”). Microbiota analysis from fecal samples collected at D10 and D14 showed that clindamycin treatment resulted in a drastic change in microbiota composition, with loss of diversity (richness (Chao index) and evenness (Shannon index)) (FIG. 2)

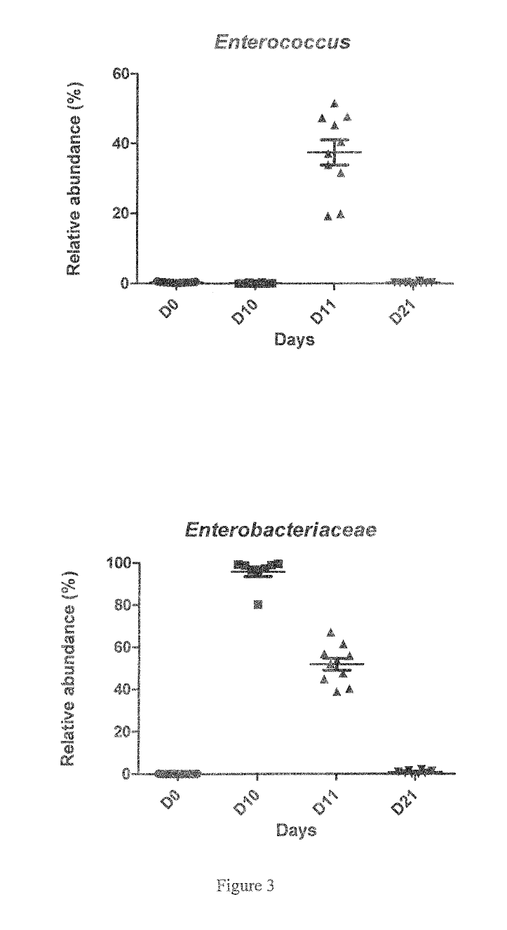

Moreover, there was a drastic increase in relative abundance of Enterococcus spp. and phylotypes belonging to Proteobacteria, specifically Enterobacteriaceae (FIG. 3).

Analysis of samples collected at D21 showed that daily consumption of L. rhamnosus CNCM I-3690 resulted in a lower extent of loss of microbial diversity (FIG. 2).

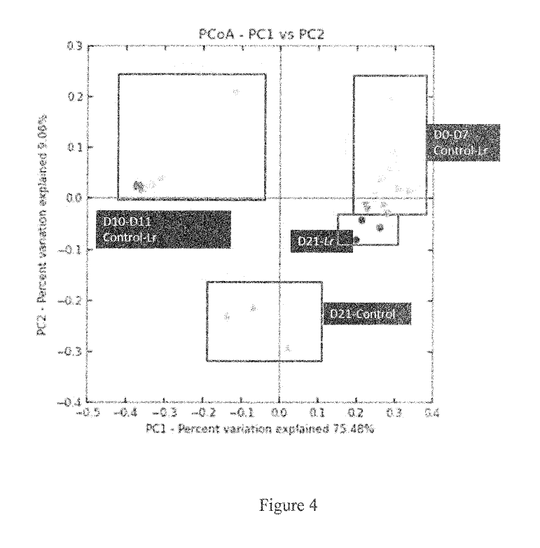

Moreover, multivariate analysis based on weighted and unweighted Unifrac distance matrices (principal Coordinate analysis) showed a clear separation between samples from DO-D7 (baseline+/−probiotic) and samples from D10-D21 (clindamycin induced dysbiosis). Notably at D21, the group of mice that received L. rhamnosus was less distinct from baseline compared to control group (FIG. 4).

SCFAs and lactate analysis at D21 from cecal contents showed that L. rhamnosus CNCM I-3690 impacted SCFAs compared to control group. The caecal butyrate/acetate ratio was significantly increased in mice receiving the L. rhamnosus strain compared to the control and also as compared to an equivalent L. paracasei-treated group (Table below) in three independent experiments (runs). In two runs, the absolute amount of butyrate was significantly increased.

butyrate/acetate ratio

RUN 1

| | | | |
|---|---|---|---|
| Control mice | 0.24 (±0.05) | | |
| | | {close oversize bracket} | p < 0.0001 |
| L. rhamnosus mice | 0.58 (±0.02) | | |
| L. paracasei mice | 0.33 (±0.05) | | |

RUN 2

| | | | |
|---|---|---|---|
| Control mice | 0.45 (±0.05) | | |
| | | {close oversize bracket} | p = 0.004 |
| L. rhamnosus mice | 0.60 (±0.10) | | |
| L. paracasei mice | 0.49 (±0.10) | | |

RUN 3

| | | | |
|---|---|---|---|
| Control mice | 0.55 (±0.25) | | |
| | | {close oversize bracket} | p = 0.003 |
| L. rhamnosus mice | 0.73 (±0.18) | | |

1. Lactobacillus rhamnosus, for use to maintain or increase the intestinal microbiota diversity of a subject.

2. Lactobacillus rhamnosus, for use to decrease the intestinal dysbiosis of a subject.

3. Lactobacillus rhamnosus, for preventing a gastrointestinal bacterial infection and/or the development of a disease caused by a pathogen in the gastrointestinal tract of a subject.

4. The Lactobacillus rhamnosus of claim 3, wherein said pathogen is Enterococcus faecalis.

5. Lactobacillus rhamnosus, for use for increasing intestinal short-chain fatty acid of a subject.

6. The Lactobacillus rhamnosus of claim 5, wherein the said Lactobacillus rhamnosus increases the level of butyrate.

7. The Lactobacillus rhamnosus of claim 5, wherein the said Lactobacillus rhamnosus increases the butyrate/acetate ratio.

8. Use according to claim 1, wherein said subject has intestinal dysbiosis.

9. The use according to claim 8, wherein said dysbiosis is antibiotic-induced dysbiosis.

10. The use according to claim 1, wherein said Lactobacillus rhamnosus is CNCM I-3690.

11. The Lactobacillus rhamnosus, for use according to claim 1, wherein said Lactobacillus rhamnosus is in an orally administrable composition.

12. The Lactobacillus rhamnosus, for use according to claim 11, wherein said composition is a fermented dairy product.
